(19) United States
(12) Patent Application Publication
(10) Pub. No.: US 2002/0183217 A1
(43) Pub. Date: Dec. 5, 2002

(54) Detergent cosmetic compositions containing an anionic surfactant derived from amino acids and salts thereof and an insoluble conditioning agent and uses thereof

(75) Inventors: Beatrice Perron, Jouy en Josas (FR); Serge Restle, Saint-Prix (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(73) Assignee: L'OREAL

(21) Appl. No.: 10/108,333

(22) Filed: Mar. 29, 2002

(30) Foreign Application Priority Data
Mar. 30, 2001 (FR) 01 04381

(51) Int. Cl. A61K 7/02; A61K 7/50

(52) U.S. Cl. 510/130; 510/136; 510/499

(57) ABSTRACT

Novel detergent cosmetic compositions comprising, in a cosmetically acceptable medium, (a) at least one anionic surfactant chosen from the N-acylated compounds of mono- and polyamidated polycarboxylic amino acids and the salts of said acids and (b) at least one insoluble conditioning agent. This combination imparts at least one of the cosmetic properties of sleekness, lightness and softness while minimizing keratinous fiber regreasing phenomenon. These compositions are used, for example, for washing and/or conditioning a keratinous material such as the hair and the skin.

The present invention relates to novel cosmetic compositions comprising, in a cosmetically acceptable medium, at least one anionic surfactant chosen from the N-acylated compounds of mono- and poly-amidated polycarboxylic amino acids and the salts of said acids and at least one particular water-insoluble conditioning agent.

The use of detergent compositions (shampoos and shower gels) based essentially on conventional surfactants of the anionic, nonionic and/or amphoteric type, but more particularly of the anionic type, is common for cleansing and/or washing the hair and/or the skin. These compositions are applied to wet hair or skin and the foam generated by massaging or rubbing with the hands allows, after rinsing with water, the removal of the various types of dirt initially present on the hair or the skin.

These base compositions indeed possess good washing power, but the intrinsic cosmetic properties attached to them remain nevertheless fairly weak, in particular because of the fact that the relatively aggressive nature of such a cleansing treatment can cause in the long term damage to the keratinous materials which is marked to a greater or lesser degree, linked in particular to the gradual removal of the lipids or proteins contained in or at the surface thereof.

Accordingly, to improve the cosmetic properties of the above detergent compositions, and more particularly of those that are intended to be applied to sensitive hair (i.e. hair which has become damaged or which has been made fragile especially under the chemical action of atmospheric agents and/or of hair treatments such as permanent waving, dyeing or bleaching) it is now customary to introduce therein additional cosmetic agents called conditioning agents, intended mainly to repair or limit the harmful or undesirable effects induced by the various treatments or attacks to which the hair fibres are subjected more or less repeatedly. These conditioning agents can of course also improve the cosmetic behaviour of natural hair.

The conditioning agents most commonly used to date in shampoos are cationic polymers, silicones and/or silicone-based derivatives, which indeed confer on washed, dry or wet hair greatly improved ease of disentanglement, softness and sleekness compared to what may be obtained with the corresponding cleansing compositions not containing them.

However, in spite of the progress recently made in the field of shampoos based on cationic polymers and/or silicone, the latter are not completely truly satisfactory, such that a high demand currently still exists in relation to being able to have novel products exhibiting better performance at the level of at least one of the cosmetic properties mentioned above.

The N-acylated anionic surfactants of mono- and poly-amidated polycarboxylic amino acids and their salts have already been recommended in detergent cosmetic compositions. They have been described, for example, in patent application WO 97/03171, the disclosure of which is hereby incorporated by reference.

The washing compositions for the hair using these surfactants alone may not lead to completely satisfactory cosmetic properties.

The aim of the invention is therefore to provide detergent cosmetic compositions having at least one improved cosmetic property, such as disentanglement, sleekness and/or softness of the hair.

However, the inventors have now found that the combination of particular insoluble conditioning agents and a particular anionic surfactant makes it possible to achieve at least one of these aims.

These novel compositions can make it possible to better deposit these insoluble conditioning agents on a keratinous material (especially the hair) than a composition containing conventional anionic surfactants such as the salts of N-cocoylglutamic acid.

The compositions in accordance with the invention confer on the keratinous material, in particular the hair, a remarkable treatment effect which manifests itself in particular by at least one of the properties of lightness, sleekness, softness, suppleness, and ease of disentanglement with minimal sensation of a charged feel.

The subject of the invention is thus a detergent cosmetic composition, characterized in that it comprises, in a cosmetically acceptable medium, at least one anionic surfactant chosen from N-acylated compounds of mono- and polyamidated polycarboxylic amino acids and the salts of said acids and at least one insoluble conditioning agent chosen from:

synthetic oils, mineral oils, and natural oils,

animal and vegetable oils,

fluorinated and perfluorinated oils,

natural and synthetic waxes,

ceramide compounds, and

carboxylic acid esters chosen from esters of monoalcohols and esters of polyols, the polyols having at least 3 carbon atoms.

Another subject of the invention relates to the use of at least one anionic surfactant chosen from the N-acylated compounds of mono- and poly-amidated polycarboxylic amino acids and the salts of said acids in, or for the manufacture of a cosmetic composition comprising at least one insoluble conditioning agent as defined above.

Another subject of the invention relates to a method for treating a keratinous material, such as hair, comprising applying to said material an effective amount of a cosmetic composition according to the invention.

A subject of the invention is also the use of a composition according to the invention in order to increase at least one of disentanglement, and sleeking of the hair, in order to impart at least one of volume, lightness, softness, suppleness and manageability on the hair.

According to the present invention, the expression keratinous material is understood to encompass the hair, the eyelashes, the eyebrows, the skin, the nails, the mucous membranes or the scalp, and as a common example, the hair.

Various aspects of the invention will now be presented in detail. All the meanings and definitions of the compounds used in the present invention given below are valid for all aspects of the invention.

N-acylated Anionic Surfactants of Mono and Polyamidated Polycarboxylic Amino Acids

The N-acylated anionic surfactants of mono- and polyamidated polycarboxylic amino acids and their salts may have, for example, the following formula (I):

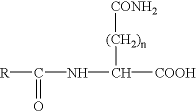

in which:

R is chosen from linear and branched, saturated and unsaturated, hydrocarbon radicals comprising from 5 to 29 carbon atoms, for example, R is chosen from mono- and polyunsaturated alkyl and alkenyl radicals comprising from 5 to 29 carbon atoms and as a further example from 7 to 22 carbon atoms, and

n is an integer chosen from 1 and 2.

An additional example would be the N-acylated compounds of mono- and polyamidated polycarboxylic amino acids of formula (I) in which R is chosen from linear, branched, and saturated alkyl radicals comprising from 7 to 29 carbon atoms, such as from 7 to 22 carbon atoms.

The salts of the compounds of formula (I) may be salts of alkali metals (for example sodium and potassium), of alkaline-earth metals (which may be, for example, calcium and magnesium), salts of aqueous ammonia, salts of amines such as those of monoethanolamine, diethanolamine, triethanolamine, 3-amino-1,2-propanediol, and ammonium salts derived from basic amino acids such as lysine, arginine, sarcosine, ornithine, and citrulline.

Among the surfactants of formula (I), there may be mentioned, for example, the salts of N-cocoylglutamine, such as the triethanolamine salt of N-cocoylglutamine and further such as the product marketed under the name FOAM UP DOUCE GM by the company KYOWA HAKKO.

According to the invention, the anionic surfactant(s) chosen from the N-acylated compounds of mono- and polyamidated polycarboxylic amino acids and their salts may, for example, represent from 1% to 30% by weight, such as from 3% to 15% by weight relative to the total weight of the final composition.

In the context of the present application, the expression conditioning agent is understood to mean any agent having as a function the improvement of at least one of the cosmetic properties of a keratinous material such as hair, in particular softness, disentanglement, feel, and static electricity.

The insoluble conditioning agents in accordance with the invention may be solid, liquid or pasty at room temperature (25° C.) and at atmospheric pressure, they may be provided in particular in the form of oils, waxes, resins or gums.

The insoluble conditioning agents are in particular dispersed in the compositions in the form of particles generally having a mean size in numerical terms of from 2 nanometers to 100 microns, for example, from 30 nanometers to 20 microns. As will be recognized by one skilled in the art, the mean size of the particles may be measured by a technique suitable for the size measurement being taken, for example, for particles essentially spherical, by scattering techniques such as static light scattering in which a laser granulometer such as MALVERN or an optical particle counter such as Coulter counter by Coultronix may be employed, and dynamic light scattering, in which a laser granulometer such as Brookhaven is employed.

The insoluble conditioning agents are insoluble in water at a concentration greater than or equal to 0.1% by weight in water at 25° C., that is to say that they do not form a macroscopically transparent isotropic solution, meaning that the solution is not homogeneous to the naked eye and, to the naked eye, has properties that vary with direction.

Oils

The synthetic oils may be polyolefins, for example, poly-α-olefins chosen from hydrogenated and nonhydrogenated polybutene, such as hydrogenated and nonhydrogenated polyisobutene.

Examples of the oligomers used in the polybutene polyolefins include isobutylene having a molecular weight of less than 1 000 and mixtures thereof with polyisobutylenes having a molecular weight greater than 1 000 such as from 1 000 to 15 000.

Examples of poly-α-olefins which can be used in the context of the invention include the polyisobutenes sold under the name PERMETHYL 99 A, 101 A, 102 A, 104 A (n=16) and 106 A (n=38) by the company PRESPERSE Inc, and the products sold under the name ARLAMOL HD (n=3) by the company ICI (n denoting the degree of polymerization).

Another example of poly-α-olefins include hydrogenated and nonhydrogenated polydecene poly-α-olefins. These products are sold, for example, under the names ETHYLFLO by the company ETHYL CORP., and ARLAMOL PAO by the company ICI.

The natural, animal, mineral and vegetable oils may be chosen, for example, from sunflower, maize, soyabean, avocado, jojoba, gourd, grapeseed, sesame, hazelnut, coconut, and fish oils, glycerol tricaprocaprylate, and vegetable and animal oils of formula R₉COOR₁₀. In formula R₉COOR₁₀ R₉ is chosen from residues of higher fatty acids comprising from 7 to 29 carbon atoms and R₁₀ is chosen from linear and branched hydrocarbon chains containing from 3 to 30 carbon atoms, such as alkyl and alkenyl groups. Specific examples include Purcellin oil and liquid jojoba wax.

It is also possible to use natural and synthetic essential oils such as, for example, eucalyptus, lavandin, lavender, vetiver, Litsea cubeba, lemon, sandalwood, rosemary, camomile, savory, nutmeg, cinnamon, hyssop, caraway, orange, geraniol, cade, and bergamot oils.

Waxes are natural (animal or plant) or synthetic substances which are solid at room temperature (20°-25° C.). They are insoluble in water, soluble in oils and are capable of forming a water-repelling film. Waxes are soluble in at least one of the following oils or in their mixtures at a concentration greater than or equal to 0.1% by weight in the oil(s) at ambient temperature (20-25 degrees C.), which is to say that they form a macroscopically transparent isotropic solution as defined above for insoluble conditioning agents. The oils are chosen from cylcomethicone, vaselin oil and C12-C15 alkyl benzoate and mixtures thereof. C12-C15 alkyl benzoate is a product marketed under the name FINCOLV TN by the company FINETEX.

Additional information on the definition of waxes may be found in P. D. Dorgan, Drug and Cosmetic Industry, December 1983, pp. 30-33.

The wax or the waxes can be chosen from carnauba wax, candelilla wax, Esparto wax, paraffin wax, ozokerite, vegetable waxes such as olive tree wax, rice wax, hydrogenated jojoba wax, absolute waxes from flowers such as the essential wax of blackcurrant flower sold by the company BERTIN (France), and animal waxes such as beeswaxes and modified beeswaxes (cerabellina). Examples of other waxes and waxy raw materials which can be used according to the invention include marine waxes such as that sold by the company SOPHIM under the reference M82 and polyethylene and polyolefin waxes in general.

Ceramide Compounds

According to the present invention, the ceramide compounds are chosen from natural ceramides, synthetic ceramides, glycoceramides, pseudoceramides, and neoceramides.

Ceramide compounds are for example described in patent applications DE4424530, DE4424533, DE4402929, DE4420736, WO95/23807, WO94/07844, EP-A-0646572, WO95/16665, FR-2 673 179, EP-A-0227994 and WO 94/07844, WO94/24097, and WO094/10131 the disclosures of which are hereby incorporated by reference.

The ceramide compounds which can, for example, be used according to the present invention correspond to the general formula (IV):

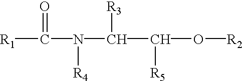

in which:

R₁ is chosen from:

saturated and unsaturated, linear and branched C₁-C₅₀, such as C₅-C₅₀ hydrocarbon radicals, it being possible for this radical to be substituted with at least one hydroxyl group optionally esterified with an acid R₇COOH, R₇ being chosen from saturated and unsaturated, linear and branched, optionally mono- and polyhydroxylated C₁-C₃₅ hydrocarbon radicals, it being possible for the hydroxyl(s) of the radical R₇ to be esterified with an acid chosen from saturated and unsaturated, linear and branched, optionally mono- and polyhydroxylated C₁-C₃₅ fatty acids;

radicals R″—(NR^{o}—CO)—R′—, wherein R^{o} is chosen from a hydrogen atom and mono- and polyhydroxylated, such as monohydroxylated, C₁-C₂₀ hydrocarbon radicals, wherein R′ and R″ are chosen from hydrocarbon radicals in which the sum of the carbon atoms is from 9 to 30;

radicals R₈—O—CO—(CH₂)ₚ—, wherein R₈ is chosen from C₁-C₂₀ hydrocarbon radicals and p is an integer ranging from 1 to 12-R₂ is chosen from a hydrogen atom, saccharide radicals, such as (glycosyl)ₙ, (galactosyl)ₘ and sulphogalactosyl radicals, sulphate and phosphate residues, phosphorylethylamine radicals and phosphorylethylammonium radicals, wherein n is an integer chosen from 1 to 4 and m is an integer chosen from 1 to 8;

R₃ is chosen from a hydrogen atom and saturated and unsaturated, monohydroxylated, polyhydroxylated and nonhydroxylated C₁-C₃₃ hydrocarbon radicals, it being possible for the hydroxyl(s) to be esterified with an acid chosen from inorganic acids and R₇COOH, R₇ having the same meanings as defined above, it being possible for the hydroxyl(s) to be esterified with an acid chosen from (glycosyl)ₙ-COOH and (galactosyl)ₘ-COOH wherein n and m are as defined above for R₂, sulphogalactosyl acid, phosphorylethylamine acid and phosphorylethylammonium acid, it being possible for said R₃ hydrocarbon radicals to be substituted with at least one C₁-C₁₄ alkyl radical, for example, R₃ can be chosen from C₁₅-C₂₆ α-hydroxyalkyl radicals, the hydroxyl group being optionally esterified with an acid chosen from C₁₆-C₃₀ α-hydroxy acids;

R₄ is chosen from a hydrogen atom, methyl and ethyl radicals, saturated and unsaturated, linear and branched, optionally hydroxylated C₃-C₅₀ hydrocarbon radicals and —CH₂—CHOH—CH₂—O—R₆ radicals wherein R₆ is chosen from C₁₀-C₂₆ hydrocarbon radicals and R₈—O—CO—(CH₂)ₚ— radicals, wherein R₈ is chosen from C₁-C₂₀ hydrocarbon radicals and p is an integer chosen from 1 to 12,

R₅ is chosen from a hydrogen atom, saturated and unsaturated, linear and branched, optionally mono- or polyhydroxylated C₁-C₃₀ hydrocarbon radicals, it being possible for the hydroxyl(s) to be esterified with an acid chosen from (glycosyl)ₙ-COOH and (galactosyl)ₘ-COOH, sulphogalactosyl acid, phosphorylethylamine acid and phosphorylethylammonium acid wherein n and m are as defined above for R₂, with the proviso that when R₃ and R₅ denote hydrogen or when R₃ denotes hydrogen and R₅ denotes methyl, then R₄ is not chosen from a hydrogen atom, and methyl and ethyl radicals.

Examples of compounds of formula (IV) include the ceramides and glycoceramides whose structure is described by DOWNING in Journal of Lipid Research Vol. 35, 2060-2068, 1994, and those described in French patent application FR-2,673,179, whose teachings are included herein by way of reference.

Further examples of ceramide compounds of formula (IV) are those for which R₁ is chosen from saturated and unsaturated, optionally hydroxylated C₁₄-C₂₂ alkyl groups such as those derived from fatty acids; R₂ denotes a hydrogen atom; R₃ is chosen from linear, optionally hydroxylated C₁₁-C₁₇, radicals such as C₁₃₋₁₅ radicals; and R₄ and R₅ are as defined above.

Examples of specific ceramide compounds include:

2-N-linoleoylaminooctadecane-1,3-diol,

2-N-oleoylaminooctadecane-1,3-diol,

2-N-palmitoylaminooctadecane-1,3-diol,

2-N-stearoylaminooctadecane-1,3-diol,

2-N-behenoylaminooctadecane-1,3-diol,

2-N-[2-hydroxypalmitoyl]aminooctadecane-1,3-diol,

2-N-stearoylaminooctadecane-1,3,4-triol,

2-N-palmitoylaminohexadecane-1,3-diol,

(bis-(N-hydroxyethyl-N-cetyl)malonamide),

N-(2-hydroxyethyl)-N-(3-cetyloxy-2-hydroxypropyl)amide of cetylic acid,

N-docosanoyl-N-methyl-D-glucamine and mixtures of these compounds.

As another example the ceramide compound can be N-stearoylphytosphingosine.

Flourinated and Perfluorinated Oils

The fluorinated and perfluorinated oils are for example the perfluoropolyethers described in patent application EP-A-486135 and the fluorohydrocarbon-containing compounds described in patent application WO 93/11103, the disclosure of which are hereby incorporated by reference.

The term fluorohydrocarbon-containing compounds is understood to mean compounds whose chemical structure comprises a carbon-containing backbone in which some hydrogen atoms have been substituted by fluorine atoms.

The fluorinated oils may also be fluorocarbons such as fluoroamines, for example perfluorotributylamine, and fluorinated hydrocarbons, for example perfluorodecahydronaphthalene, fluoroesters and fluoroethers.

The perfluoropolyethers are for example sold under the trade names FOMBLIN by the company MONTEFLUOS and KRYTOX by the company DU PONT.

Among the fluorohydrocarbon-containing compounds, there may also be mentioned fluorinated fatty acid esters such as the product sold under the name NOFABLE FO by the company NIPPON OIL.

According to the invention, the esters may be liquid at a temperature less than or equal to 30° C.

The esters of monoalcohols are in particular esters of acids chosen from C₂-C₂₆ mono-, di-, tri- and tetracarboxylic acids and of alcohols chosen from C₁-C₂₆ monoalcohols, the total number of carbons in the esters being, for example, greater than or equal to 10 and less than 80.

The monocarboxylic acid esters are, for example, monoesters of acids chosen from saturated and unsaturated, linear and branched C₂-C₂₆ aliphatic acids and of alcohols chosen from saturated and unsaturated, linear and branched C₁-C₂₆ aliphatic monoalcohols, wherein the total number of carbons in the esters is greater than or equal to 10.

Examples of these monoesters include dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates; 2-ethylhexyl palmitate; 2-octyldecyl palmitate; alkyl myristates, such as isopropyl, butyl, cetyl and 2-octyldodecyl myristate, hexyl stearate; butyl stearate; isobutyl stearate; dioctyl malate; hexyl laurate; and 2-hexyldecyl laurate.

It is also possible to use esters chosen from esters of acids chosen from C₄-C₂₂ di- and tricarboxylic acids and of alcohols chosen from C₁-C₂₂ alcohols. Examples include diethyl sebacate; diisopropyl sebacate; diisopropyl adipate; di-n-propyl adipate; dioctyl adipate; diisostearyl adipate; dioctyl maleate; octyldodecyl stearoyl stearate; tridecyl erucate; triisopropyl citrate; triisostearyl citrate; trioctyldodecyl citrate; and trioleyl citrate.

The esters of polyols having at least 3 carbon atoms are for example chosen from the esters of acids chosen from C₂-C₂₆ mono-, di- and tricarboxylic acids and of alcohols chosen from C₃-C₂₆ di-, tri, tetra- and pentahydroxy alcohols. Examples include glyceryl undecylenate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; glyceryl trilactate; and glyceryl trioctanoate.

Further examples of the abovementioned esters include ethyl and isopropyl palmitates; 2-ethylhexyl palmitate; 2-octyldecyl palmitate; alkyl myristates such as isopropyl, butyl and 2-octyldodecyl myristate; butyl stearate such as isobutyl stearate; dioctyl malate; hexyl laurate; isononyl isononanoate and cetyl octanoate.

According to the invention,the insoluble conditioning agent(s) may, for example, represent from 0.001% to 20% by weight, as a further example from 0.01% to 10% by weight, such as from 0.1 to 3% by weight, relative to the total weight of the final composition.

The compositions of the invention may also contain, in addition, at least one other surfactant which is generally, for example, present in a quantity of from 0.1% to 40% by weight approximately, as a further example from 3% to 30% such as from 5% to 20%, relative to the total weight of the composition.

This surfactant may be chosen from anionic, amphoteric and nonionic surfactants, and mixtures thereof.

Additional surfactants for carrying out the present invention can be chosen from the following:

(i) Anionic Surfactant(s)

Their nature may not be of critical importance within the context of the present invention.

Thus, by way of example of anionic surfactants that can be employed, by themselves or as mixtures, in the context of the present invention, there may be mentioned (nonlimiting list) the salts (for example alkali metal salts such as sodium, ammonium salts, amine salts, amino alcohol salts and magnesium salts) of compounds chosen from alkyl sulphates, alkyl ether sulphates, alkylamido ether sulphates, alkylaryl-polyether sulphates, monoglyceride sulphates, alkyl sulphonates, alkyl phosphates, alkylamidesulphonates, alkyl aryl sulphonates, α-olefinsulphonates, paraffin-sulphonates, alkyl sulphosuccinates, alkyl ether sulphosuccinates, alkylamidesulphosuccinates, alkyl sulphosuccinamates, alkyl sulphoacetates, alkyl ether phosphates, acyl sarcosinates, acyl isethionates and N-acyltaurates, the alkyl and acyl radicals of all these different compounds containing from 8 to 24 carbon atoms, and the aryl radical chosen from phenyl and a benzyl group. Among the anionic surfactants which may be used in the invention there may also be mentioned the salts of acids chosen from oleic, ricinoleic, palmitic and stearic acids, acids of copra oil and of hydrogenated copra oil, and acyl lactylates in which the acyl radical contains 8 to 20 carbon atoms. Weakly anionic surfactants which may also be employed include those chosen from alkyl-D-galactosideuronic acids and the salts of said acids, polyoxyalkylenated carboxylic (C₆-C₂₄)alkyl ether acids, polyoxyalkylenated carboxylic (C₆-C₂₄)alkylaryl ether acids, polyoxyalkylenated carboxylic (C₆-C₂₄)alkyl amidoether acids and the salts of said acids, such as, for examples, salts of acids containing from 2 to 50 ethylene oxide groups, and mixtures thereof.

Additional examples of anionic surfactants include those chosen from the salts of alkyl sulphates, the saltsof alkyl ether sulphates and mixtures thereof.

(ii) Nonionic Surfactant(s)

The nonionic surfactants themselves are also compounds which are well known per se (in this respect see the “Handbook of Surfactants” by M. R. Porter, published by Blackie & Son (Glasgow and London), 1991, pp. 116-178) and, in the context of the present invention, their nature may not be of critical importance. They can thus be chosen from (nonlimiting list) alcohols, alpha-diols, alkylphenols and polyethoxylated, polypropoxylated and polyglycerolated fatty acids which have a fatty chain containing, for example, 8 to 18 carbon atoms, it being possible for the number of ethylene oxide and propylene oxide groups to each range from 2 to 50 and it being possible for the number of glycerol groups to range from 2 to 30. The copolymers of ethylene oxide and propylene oxide and the condensates of ethylene oxide and propylene oxide with fatty alcohols may also be mentioned by way of example. Further examples are chosen from polyethoxylated fatty amides containing, for example, from 2 to 30 mol of ethylene oxide, polyglycerolated fatty amides on average containing 1 to 5 glycerol groups, such as 1.5 to 4, polyethoxylated fatty amines containing, for example, 2 to 30 mol of ethylene oxide, oxyethylenated fatty acid esters of sorbitan containing from 2 to 30 mol of ethylene oxide, fatty acid esters of sucrose, fatty acid esters of polyethylene glycol, alkylpolyglycosides, N-alkylglucamine derivatives, amine oxides such as the oxides of (C₁₀-C₁₄) alkylamines, and N-acylaminopropylmorpholine oxides. Alkylpolyglycosides are still another example of nonionic surfactants for the present invention.

(iii) Amphoteric Surfactant(s)

The additional amphoteric surfactants, the nature of which may not be of critical importance in the context of the present invention, may be chosen from (nonlimiting list) derivatives of aliphatic secondary and tertiary amines in which the aliphatic radical is chosen from linear and branched chains containing 8 to 22 carbon atoms and containing at least one water-solubilizing anionic group (for example carboxylate, sulphonate, sulphate, phosphate and phosphonate); (C₈-C₂₀)alkylbetaines, sulphobetaines, (C₈-C₂₀)alkylamido(C₁-C₆)alkylbetaines and (C₈-C₂₀)alkylamido(C₁-C₆)alkylsulphobetaines.

Among the amine derivatives there may be mentioned the products sold under the name Miranol, as described in Patents U.S. Pat. Nos. 2,528,378 and 2,781,354 and of structures chosen from:

R₂—CONHCH₂CH₂—N(R₃)(R₄)(CH₂COO—) (2)

in which:

R₂ is chosen from alkyl radicals of R₂—COOH acids present in hydrolysed copra oil, such as heptyl, nonyl and undecyl radicals; R₃ is a beta-hydroxyethyl group; and R₄ is a carboxymethyl group and

R₅—CONHCH₂CH₂—N(B)(C) (3)

in which:

(B) is chosen from —CH₂CH₂OX′;

(C) is chosen from —(CH₂)_{z}—Y′, wherein z is an integer chosen from 1 and 2;

X′ is chosen from a hydrogen atom and —CH₂CH₂—COOH;

Y′ is chosen from —COOH and —CH₂—CHOH—SO₃H; and

R₅ is chosen from alkyl radicals of a carboxylic acid present in copra oil and in hydrolysed linseed oil such as alkyl radicals chosen from C₇, C₉, C₁₁, C₁₃ and C₁₇ alkyl radicals, iso C₁₇ alkyl radicals and unsaturated C₁₇ radicals.

These compounds are classified in the CTFA dictionary, 5th edition, 1993, under the names Disodium Cocoamphodiacetate, Disodium Lauroamphodiacetate, Disodium Capryloamphodiacetate, Disodium Caproamphodiacetate, Disodium Cocoamphodipropionate, Disodium Lauroamphodipropionate, Disodium Caproampho-dipropionate, Disodium Capryloamphodipropionate, Lauro-amphodipropionic acid, and Cocoamphodipropionic acid.

By way of example, there may be mentioned the product of disodium cocoamphodiacetate marketed under the trade name MIRANOL® C2M concentrate by the company RHODIA CHIMIE.

Thus, representative compositions of the invention may include mixtures of surfactants chosen from mixtures of anionic surfactants, mixtures of anionic surfactants and amphoteric surfactants and mixtures of anionic surfactants and nonionic surfactants.

Examples of such mixtures of surfactants include additional anionic surfactants chosen from sodium, triethanolamine and ammonium (C₁₂-C₁₄)alkyl sulphates, oxyethylenated sodium, triethanolamine and ammonium (C₁₂-C₁₄)alkyl ether sulphates containing 2.2 mol of ethylene oxide, and mixtures of sodium cocoyl isethionate and sodium (C₁₄-C₁₆)alphaolefin sulphonate with an amphoteric surfactant chosen from:

amphoteric surfactants such as the amine derivatives called disodium cocoamphodipropionate and sodium cocoamphopropionate marketed by the company RHODIA CHIMIE under the trade name “MIRANOL® C2M CONC” in aqueous solution at 38% of active material and under the name MIRANOL® C32

amphoteric surfactants such as alkylbetaines, for example cocobetaine marketed under the name “DEHYTON® AB 30” in aqueous solution at 32% AM (active material) by the company HENKEL and the (C₈-C₂₀)alkylamido(C₁-C₆)alkylbetaines, such as TEGOBETAINE® F 50 marketed by the company GOLDSCHMIDT.

Anionic surfactant(s) different from the N-acylated compounds of mono- and polyamidated polycarboxylic amino acids according to the invention are generally, for example, present in an amount of 1 to 30% by weight, for example 3 to 15% by weight, relative to the total weight of the composition.

The amphoteric and nonionic surfactant(s) are generally, for example, present in an amount of 0.5 to about 15% by weight, for example from 1 to 5% by weight, relative to the total weight of the composition.

The quantity and the quality of the surfactants are those sufficient to confer at least one of the qualities of satisfactory foaming and satisfactory detergent power on the final composition.

In the composition according to the present invention, all the detergent surfactants generally, for example, represent from 4 to 50% by weight, as further example, from 6 to 35% by weight and as an even further example from 8 to 25% by weight, relative to the total weight of the composition.

The composition of the invention may also contain at least one additive chosen from thickeners, perfumes, pearlescent agents, preservatives, sunscreens, silicones, anionic, nonionic and amphoteric polymers, cationic polymers, proteins, protein hydrolysates, linear and branched chain C₁₆-C₄₀ acids such as 18-methyleicosanoic acid, hydroxy acids, vitamins, provitamins such as panthenol, vegetable oils, mineral oils, synthetic oils, antidandruff agents and any other additive conventionally used in the cosmetic field which does not affect the stability and the properties of the compositions according to the invention.

These additives may be present in the composition according to the invention in proportions which may, for example, range from 0.001 to 50% by weight relative to the total weight of the composition. The precise quantity of each additive is easily determined by a person skilled in the art according to its nature and its function.

The cosmetically acceptable medium may consist solely of water or the cosmetically acceptable medium may comprise a mixture of solvents chosen from water and cosmetically acceptable solvents, for example, a lower C₁-C₄ alcohol chosen from ethanol, isopropanol, tert-butanol, and n-butanol and alkylene glycols such as propylene glycol and glycol ethers. For example, the composition may comprise from 50 to 95% by weight of water relative to the total weight of the composition.

The detergent compositions according to the invention have a final pH which is generally, for example, from 3 to 10, for further example, a pH from 4 to 8. The adjustment of the pH to the desired value may be carried out conventionally by addition of a base (organic or inorganic) to the composition, for example a base chosen from aqueous ammonia and primary, secondary and tertiary (poly)amines such as monoethanolamine, diethanolamine, triethanolamine, isopropanolamine and 1,3-propanediamine. The adjustment of the pH to the desired value may also be carried out conventionally by addition of an acid, for example a carboxylic acid such as citric acid.

The compositions in accordance with the invention may contain, in addition to the combination defined above, viscosity-regulating agents chosen from electrolytes and thickening agents which may be chosen from sodium chloride, scleroglucans, xanthan gums, fatty acid alkanolamides, alkyl ether carboxylic acid alkanolamides which are optionally oxyethylenated with up to 5 mol of ethylene oxide, such as the product marketed under the name “AMINOL A15” by the company CHEM Y, crosslinked polyacrylic acids and crosslinked acrylic acid/C₁₀-C₃₀ alkyl acrylate copolymers. These viscosity-regulating agents are used in the compositions according to the invention in proportions which may be up to 10% by weight relative to the total weight of the composition.

The compositions in accordance with the invention may also contain up to 5% of pearlescent and opacifying agents well known in the state of the art, such as those chosen from sodium and magnesium palmitates, sodium and magnesium stearates and hydroxystearates, fatty chain-containing acylated derivatives such as ethylene glycol and polyethylene glycol monostearates and distearates, and fatty chain-containing ethers such as distearyl ether and 1-(hexadecyloxy)-2-octadecanol.

The compositions according to the invention may also contain foam synergists chosen from C₁₀-C₁₈ 1,2-alkanediols and fatty alkanolamides derived from mono- and diethanolamine.

The compositions in accordance with the invention may be used for washing and treating a keratinous material chosen from hair, skin, eyelashes, eyebrows, nails, lips, and scalp, and a common example is the hair.

The detergent compositions according to the invention may be chosen, for example, from shampoos, shower gels and foam baths. The compositions of the invention may also be provided in forms chosen from rinse-off and leave-in-after-shampoos, compositions for permanent waving, straightening, dyeing and bleaching, and rinse-off compositions for permanent waving, straightening, dyeing and bleaching, which my be applied at times chosen from before and after dyeing, bleaching, permanent waving and straightening and between the two stages of permanent waving and straightening. The compositions of the invention may also be provided in the form of make-up removing products.

The compositions according to the invention may be provided in forms chosen from gels, milks, creams, emulsions, thickened lotions, and thickened foams and may be used for a keratinous material chosen from skin, scalp, nails, eyelashes, lips and hair.

These detergent compositions may be foaming and the foaming power of the compositions according to the invention, characterized by a foam height, is generally greater than 75 mm, such as greater than 100 mm measured according to the modified ROSS-MILES method (NF T 73-404/ISO696).

The modifications of the method are the following: The measurement is carried out at the temperature of 22° C. with osmosed water. The concentration of the solution is 2 g/l. The drop height is 1 m. The quantity of composition which drops is 200 ml. These 200 ml of composition fall into a measuring cylinder having a diameter of 50 mm and containing 50 ml of the composition tested. The measuring is made 5 minutes after stopping the flow of the composition.

The subject of the invention is also a method for treating a keratinous material, such as skin and hair, characterized in that it comprises applying to the keratinous material an effective amount of a cosmetic composition as defined above, and then optionally rinsing off, with water, for example.

Thus, the method according to the invention allows for at least one of treatment of a keratinous material, care of a keratinous material, washing of a keratinous material, and removal of make-up from a keratinous material such as skin and hair.

In the text which follows or in the preceding text, the percentages expressed are by weight. The invention will now be illustrated more fully with the aid of the following examples which cannot be considered as limiting the embodiments described. In the examples, AS means active substance.

### EXAMPLE 1

Three shampoo compositions were prepared, one in accordance with the invention (composition A) and the other 2 comparatives (compositions B and C):

| | A | | |
|---|---|---|---|
| | (invention) | B | C |
| Triethanolamine salt of N-cocoyl | 16.7 g | — | — |
| glutamine at 30% AS (Foam up | (5 gAS) | | |
| doucé GM from KYOWA | | | |
| HAKKO) | | | |
| Cocoylbetaine as an aqueous | 16.7 g | 16.7 g | 16.7 g |
| solution at 30% AS (DEHYTON | (5 gAS) | (5 gAS) | (5 gAS) |
| AB 30 from COGNIS) | | | |
| Sodium (C₁₂-C₁₄)alkyl ether | 57 g | 57 g | 77 g |
| sulphate oxyethylenated with 2.2 | (14.8 gAS) | (14.8 | (20 gAS) |
| mol of ethylene oxide as an | | gAS) | |
| aqueous solution at 26% of AS | | | |
| N-cocoyl glutamate as an aqueous | — | 16.7 gAS | — |
| solution at 30% AS (Acyl | | (5 gAS) | |
| glutamate CT12 from | | | |
| AJINOMOTO) | | | |
| Isopropyl myristate | 4 g | 4 g | 4 g |
| Preservatives | Qs | qs | qs |
| pH agent qs pH | 6.5 | 6.5 | 6.5 |
| Demineralized water qs | 100 g | 100 g | 100 g |

Shampooing was performed by applying about 1 g of composition A to locks of 2.5 g of previously wet sensitized hair. The shampoo was caused to lather, an exposure time of 10 minutes was allowed to elapse and then the locks were abundantly rinsed off with water. The locks were dried for 10 minutes at 60° C. A second application of the composition was carried out.

The same procedure as above is used with the comparative compositions B and C.

Experts compared the locks in pairs.

Composition A/Composition C

A panel of experts evaluated the appearance of the wet hair.

90% of the experts indicated that the hair treated with Composition A according to the invention was significantly sleeker than that treated with Composition C.

Composition A/Composition B

A panel of experts evaluated the appearance of the dry hair.

90% of the experts indicated that the hair treated with Composition A according to the invention was significantly sleeker and/or softer than that treated with Composition B.

We claim:

1. A detergent cosmetic composition comprising, in a cosmetically acceptable medium, (a) at least one anionic surfactant chosen from N-acylated compounds of mono- and polyamidated polycarboxylic amino acids and the salts of said acids and (b) at least one insoluble conditioning agent chosen from:
synthetic oils,
mineral oils,
vegetable oils,
animal oils,
natural oils,
fluorinated and perfluorinated oils,
natural and synthetic waxes,
ceramide compounds, and
carboxylic acid esters chosen from esters of monoalcohols and esters of polyols, wherein the polyols have at least 3 carbon atoms.

2. The composition of claim 1 wherein said at least one anionic surfactant is chosen from N-acylated compounds of mono- and polyamidated polycarboxylic amino acids of the following formula (I): 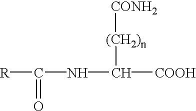
wherein:
R is chosen from linear and branched, saturated and unsaturated hydrocarbon radicals comprising from 5 to 29 carbon atoms, and
n is an integer chosen from 1 and 2.

3. The composition of claim 2, wherein R is chosen from mono- and polyunsaturated alkyl and alkenyl radicals comprising from 5 to 29 carbon atoms.

4. The composition of claim 3 wherein the alkyl and alkenyl radicals comprise from 7 to 22 carbon atoms.

5. The composition of claim 1, wherein said at least one anionic surfactant is an N-cocoylglutamine salt.

6. The composition of claim 1 wherein the synthetic oils are polyolefins chosen from hydrogenated polybutenes, nonhydrogenated polybutenes, hydrogenated polydecenes and nonhydrogenated polydecenes.

7. The composition of claim 1 wherein the animal, natural, synthetic and vegetable oils are chosen from sunflower oils, maize oils, soyabean oils, coconut oils, avocado oils, jojoba oils, gourd oils, grapeseed oils, sesame oils, hazelnut oils, fish oils, glycerol tricaprocaprylate oils, natural and synthetic essential oils, and vegetable and animal oils of formula R₉COOR₁₀ wherein R₉ is chosen from residues of higher fatty acids comprising from 7 to 29 carbon atoms and R₁₀ is chosen from linear and branched hydrocarbon chains comprising from 3 to 30 carbon atoms.

8. The composition of claim 7 wherein said hydrocarbon chains of R₁₀ are chosen from alkyl and alkenyl.

9. The composition of claim 7 wherein said natural and synthetic oils are chosen from natural and synthetic essential oils chosen from eucalyptus oils, lavandin oils, lavender oils, vetiver oils, Litsea cubeba oils, lemon oils, sandalwood oils, rosemary oils, camomile oils, savory oils, nutmeg oils, cinnamon oils, hyssop oils, caraway oils, orange oils, geraniol oils, cade oils and bergamot oils.

10. The composition of claim 1 wherein the natural and synthetic waxes are chosen from carnauba wax, candelilla wax, Esparto wax, paraffin wax, ozokerite, vegetable waxes, absolute waxes from flowers, animal waxes, marine waxes, polyethylene waxes and polyolefin waxes.

11. The composition of claim 10 wherein the absolute waxes from flowers are chosen from the essential wax of blackcurrant flower.

12. The composition of claim 10 wherein the animal waxes are chosen from beeswaxes.

13. The composition of claim 10 wherein the vegetable waxes are chosen from olive tree wax, rice wax, and hydrogenated jojoba wax.

14. The composition of claim 1 wherein the ceramide compounds are chosen from the formula (IV): 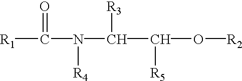
in which:
R₁ is chosen from:
(A) saturated and unsaturated, linear and branched C₁-C₅₀ hydrocarbon radicals, wherein the C₁-C₅₀ hydrocarbon radicals are optionally substituted with at least one hydroxyl group and optionally esterified with an acid of formula R₇COOH, wherein
R₇ is chosen from saturated and unsaturated, linear and branched, optionally substituted mono- and polyhydroxylated C₁-C₃₅ hydrocarbon radicals;
(B) radicals R″—(NR^{o}—CO)—R′—, wherein R^{o} is chosen from hydrogen and mono- and polyhydroxylated C₁-C₂₀ hydrocarbon radicals; and R′ and R″ are hydrocarbon radicals wherein together R′ and R″ comprise a total of 9 to 30 carbon atoms;
(C) R₈—O—CO—(CH₂)ₚ—, wherein R₈ is chosen from C₁-C₂₀ hydrocarbon radicals, and
p is chosen from an integer from 1 to 12;
R₂ is chosen from hydrogen, saccharide radicals, sulphate and phosphate residues, phosphorylethylamine radicals and phosphorylethylammonium radicals;
R₃ is chosen from hydrogen, saturated and unsaturated, monohydroxylated, polyhydroxylated and nonhydroxylated C₁-C₃₃ hydrocarbon radicals, wherein the hydroxyl(s) of the C₁-C₃₃ hydrocarbon radicals are optionally esterified with an acid chosen from inorganic acids and R₇COOH, wherein R₇ is chosen from saturated and unsaturated, linear and branched, optionally substituted mono- and polyhydroxylated C₁-C₃₅ hydrocarbon radicals;
R₄ is chosen from hydrogen, methyl and ethyl radicals, saturated and unsaturated, linear and branched, optionally hydroxylated C₃-C₅₀ hydrocarbon radicals and —CH₂—CHOH—CH₂—O—R₆ wherein R₆ is chosen from C₁₀-C₂₆ hydrocarbon radicals and R₈—O—CO—(CH₂)ₚ— wherein R₈ is chosen from C₁-C₂₀ hydrocarbon radicals and p is an integer chosen from 1 to 12; and
R₅ is chosen from hydrogen, saturated and unsaturated, linear and branched, optionally mono- and polyhydroxylated C₁-C₃₀ hydrocarbon radicals, wherein the hydroxyl(s) of the mono- and polyhydroxylated C₁-C₃₀ hydrocarbon radicals can be esterified with an acid chosen from (glycosyl)ₙ-COOH, (galactosyl)ₘ-COOH, sulphogalactosyl acid, phosphorylethylamine acid and phosphorylethylammonium acid;
provided, however, that when R₃ and R₅ are hydrogen or when R₃ is hydrogen and R₅ is methyl, then R₄ is not chosen from a hydrogen atom, methyl and ethyl radicals.

15. The composition of claim 14 wherein the hydroxyl(s) of the mono- and polyhydroxylated C₁-C₃₅ hydrocarbon radicals of the radical R₇ in R₁ are esterified with an acid chosen from saturated and unsaturated, linear and branched, mono- and polyhydroxylated C₁-C₃₅ fatty acids.

16. The composition of claim 14 wherein the saccharide radicals of R₃ are chosen from (glycosyl)ₙ, (galactosyl)ₘ and sulphogalactosyl radicals, wherein n is an integer from 1 to 4 and m is an integer from 1 to 8.

17. The composition of claim 14 wherein the hydroxyl(s) of the mono- and polyhydroxylated C₁-C₃₅ hydrocarbon radicals of the radical R₇ in R₃ are etherified with a radical chosen from (glycosyl)ₙ-COOH, (galactosyl)ₘ-COOH, sulphogalactosyl acid, phosphorylethylamine acid and phosphorylethylammonium acid radicals wherein n is an integer from 1 to 4 and m is an integer from 1 to 8.

18. The composition of claim 14 wherein R₃ is substituted with at least one C₁-C₁₄ alkyl radical;

19. The composition of claim 14 wherein R₃ is chosen from C₁₅-C₂₆ α-hydroxyalkyl radicals, wherein the hydroxyl group is optionally esterified with a C₁₆-C₃₀ α-hydroxy acid.

20. The composition of claim 1 wherein the ceramide compounds are chosen from:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol,
2-N-palmitoylaminohexadecane-1,3-diol,
(bis-(N-hydroxyethyl-N-cetyl)malonamide),
N-(2-hydroxyethyl)-N-(3-cetyloxy-2-hydroxypropyl)amide of cetylic acid, and
N-docosanoyl-N-methyl-D-glucamine.

21. The composition of claim 1 wherein the ceramide compounds are chosen from N-stearoylphytosphingosine.

22. The composition of claim 1 wherein the carboxylic acid esters are liquid at a temperature of less than or equal to 30° C.

23. The composition of claim 1 wherein said esters of monoalcohols are chosen from esters of acids chosen from mono-, di-, tri- and tetracarboxylic acids and of alcohols chosen from monoalcohols.

24. The composition of claim 23, wherein said acids are chosen from C₂-C₂₆ mono-, di-, tri- and tetracarboxylic acids and said alcohols are chosen from C₁-C₂₆ monoalcohols.

25. The composition of claim 1 wherein said esters of polyols are chosen from esters of acids chosen from mono-, di- and tricarboxylic acids and of alcohols chosen from C₃-C₂₆ di-, tri-, tetra- and pentahydroxy alcohols.

26. The composition of claim 25, wherein said acids are chosen from C₂-C₂₆ mono-, di-, tri- and tetracarboxylic acids

27. The composition of claim 1 wherein the carboxylic acid esters are chosen from ethyl and isopropyl palmitates, 2-ethylhexyl palmitate, 2-octyldecyl palmitate, alkyl myristates, butyl stearate, isobutyl stearate; dioctyl malate, hexyl laurate, isononyl isononanoate, and cetyl octanoate.

28. The composition of claim 27 wherein the alkyl myristates are selected from isopropyl, butyl, cetyl and 2-octyldodecyl myristate.

29. The composition of claim 1 wherein the at least one anionic surfactant is present at a concentration of from 1 to 30% by weight relative to the total weight of the composition.

30. The composition of claim 1 wherein the at least one anionic surfactant is present at a concentration of from 3 to 15% by weight relative to the total weight of the composition.

31. The composition of claim 1 wherein said at least one insoluble conditioning agent is present at a concentration of from 0.001% to 10% by weight relative to the total weight of the composition.

32. The composition of claim 1 wherein said at least one insoluble conditioning agent is present at a concentration of from 0.005% to 5% by weight relative to the total weight of the composition.

33. The composition of claim 1 wherein said at least one insoluble conditioning agent is present at a concentration of from 0.01% to 3% by weight relative to the total weight of the composition.

34. The composition of claim 1 wherein the composition further comprises at least one additional surfactant chosen from anionic, cationic, nonionic and amphoteric surfactants.

35. The composition of claim 34, wherein the at least one additional surfactant is present at a concentration of from 0.5% to 40% by weight relative to the total weight of the composition.

36. The composition of claim 34, wherein the at least one additional surfactant is present at a concentration of from 3% to 30% by weight relative to the total weight of the composition.

37. The composition of claim 34, wherein the at least one additional surfactant is present at a concentration of from 5% to 20% by weight relative to the total weight of the composition.

38. The composition of claim 1 wherein said composition further comprises at least one additive chosen from thickeners, perfumes, pearlescent agents, preservatives, sunscreens, silicones, anionic, non-ionic, and amphoteric polymers, cationic polymers, proteins, protein hydrolysates, linear and branched chain C₁₆-C₄₀ acids, hydroxy acids, vitamins, provitamins, antidandruff agents and mixtures thereof.

39. The composition of claim 38 wherein said linear and branched chain C₁₆-C₄₀ acids are chosen from 18-methyleicosanoic acid.

40. The composition of claim 38 wherein said provitamins are chosen from panthenol.

41. A shampoo, a washing composition for the skin, a rinse-off or leave-in after-shampoo, a composition for permanent waving, straightening, dyeing or bleaching, or a rinse-off composition for permanent waving, straightening, dyeing or bleaching comprising, in a cosmetically acceptable medium, (a) at least one anionic surfactant chosen from N-acylated compounds of mono- and polyamidated polycarboxylic amino acids and the salts of said acids and (b) at least one insoluble conditioning agent chosen from:
synthetic oils,
mineral oils,
vegetable oils,
animal oils,
natural oils,
fluorinated and perfluorinated oils,
natural and synthetic waxes,
ceramide compounds, and
carboxylic acid esters chosen from esters of monoalcohols and esters of polyols, wherein the polyols have at least 3 carbon atoms.

42. A method for washing a keratinous material comprising applying to said keratinous material an effective amount of a composition comprising, in a cosmetically acceptable medium, (a) at least one anionic surfactant chosen from N-acylated compounds of mono- and polyamidated polycarboxylic amino acids and the salts of said acids and (b) at least one insoluble conditioning agent chosen from:
synthetic oils,
mineral oils,
vegetable oils,
animal oils,
natural oils,
fluorinated and perfluorinated oils,
natural and synthetic waxes,
ceramide compounds, and
carboxylic acid esters chosen from esters of monoalcohols and esters of polyols, wherein the polyols have at least 3 carbon atoms.

43. The method according to claim 42, wherein said keratinous material is hair.

44. A method for increasing the disentanglement or sleeking of hair comprising applying to the hair an effective amount of a composition and then optionally rinsing off, wherein said composition comprises, in a cosmetically acceptable medium, (a) at least one anionic surfactant chosen from N-acylated compounds of mono- and polyamidated polycarboxylic amino acids and the salts of said acids and (b) at least one insoluble conditioning agent chosen from:
synthetic oils,
mineral oils,
vegetable oils,
animal oils,
natural oils,
fluorinated and perfluorinated oils,
natural and synthetic waxes,
ceramide compounds, and
carboxylic acid esters chosen from esters of monoalcohols and esters of polyols, wherein the polyols have at least 3 carbon atoms,
wherein said applying occurs for a time sufficient to impart at least one of volume, lightness, softness, suppleness and manageability to the hair.

45. A method for treating a keratinous material comprising applying to the material a cosmetic composition and then optionally rinsing off, the cosmetic composition comprising, in a cosmetically acceptable medium, (a) at least one anionic surfactant chosen from N-acylated compounds of mono- and polyamidated polycarboxylic amino acids and the salts of said acids and (b) at least one insoluble conditioning agent chosen from:
synthetic oils,
mineral oils,
vegetable oils,
animal oils,
natural oils,
fluorinated and perfluorinated oils,
natural and synthetic waxes,
ceramide compounds, and
carboxylic acid esters chosen from esters of monoalcohols and esters of polyols, wherein the polyols have at least 3 carbon atoms.

46. The method of claim 45 wherein said keratinous material is hair.
